(19) United States
(12) Patent Application Publication
(10) Pub. No.: US 2021/0307947 A1
(43) Pub. Date: Oct. 7, 2021

(54) OVER MOLDED IMPLANTABLE DEVICE TO PROTECT TUBING FROM PUNCTURE

(71) Applicant: Allergan, Inc., Irvine, CA (US)

(72) Inventors: Ethan Franklin, Goleta, CA (US); Justin Schwab, Santa Barbara, CA (US)

(73) Assignee: Allergan, Inc., Irvine, CA (US)

(21) Appl. No.: 13/972,801

(22) Filed: Aug. 21, 2013

Related U.S. Application Data
(63) Continuation of application No. 13/161,399, filed on Jun. 15, 2011, now abandoned.
Application No. 13/161,399 is a continuation-in-part of application No. 13/019,238, filed on Feb. 1, 2011, now abandoned.
Application No. 13/019,238 is a continuation-in-part of application No. 12/771,609, filed on Apr. 30, 2010, now Pat. No. 8,992,415.

(51) Int. Cl. A61F 5/00; A61M 39/02

(52) U.S. Cl. CPC A61F 5/0056; A61M 2039/1066; A61M 2039/0226; A61M 39/0208

(57) ABSTRACT

An implantable device used in a gastric band system includes an access port, a tube coupled to the access port, and a shielding device covering a portion of the tube. The shielding device is positioned adjacent to the access port and covers the end of the tube coupled to the access port. The shielding device is made from a puncture resistant material, to protect the tube from puncture by a misplaced syringe needle inserted by a physician.

### RELATED APPLICATIONS

This application is a continuation of U.S. patent application Ser. No. 13/161,399, filed Jun. 15, 2011, which is a continuation-in-part of U.S. patent application Ser. No. 13/019,238, filed on Feb. 1, 2011, which is a continuation-in-part of U.S. patent application Ser. No. 12/771,609, filed on Apr. 30, 2010, the entire disclosures of which applications are incorporated herein by reference in their entireties.

### FIELD

The present invention generally relates to medical systems and apparatus and uses thereof for treating obesity and/or obesity-related diseases, and more specifically, relates to an implantable device used in a medical system to protect tubing from puncture.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts the food's passage into a lower portion of the stomach. When the stoma is of an appropriate size that is restricted by a gastric band, food held in the upper portion of the stomach provides a feeling of satiety or fullness that discourages overeating. Unlike gastric bypass procedures, gastric band apparatus are reversible and require no permanent modification to the gastrointestinal tract.

Certain types of gastric band systems may operate through a hydraulic force. The size of the band placed around the stomach may depend on the volume of the fluid in the band. An access port may be used to control the amount of fluid in the band. The access port may be located below the surface of an individual's skin. The physician accesses the access port to either increase or decrease the amount of fluid in the band. The physician inserts a long hypodermic needle through the surface of the skin and into the access port. The physician may then deposit or remove fluid from the system to control operation of the gastric band. However, the access port may be under many layers of fat, and may be difficult to locate. If the physician cannot properly locate the access port, the physician may improperly insert the hypodermic needle into the individual's body.

If the physician improperly inserts the hypodermic needle into the individual's body, the hypodermic needle may puncture the tube leading from the access port to the gastric band. The tube contains fluid that may leak causing the gastric band to eventually fail. The entire gastric band system may then need to be removed from the individual's body, or the physician may need to perform an operation to mend the punctured tube.

### SUMMARY

Generally described herein is an implantable shielding device that protects tubing used in a gastric band system. A protective system placed over the tubing may protect the tube from errant needle sticks.

In one embodiment, the implantable device comprises an access port configured to attach to body tissue, a tube coupled to the access port, and a shielding device coupled to the tube. The shielding device is positioned adjacent to the access port and covers the end of the tube coupled to the access port. The shielding device is made from a puncture resistant material. The shielding device protects the tube from puncture, by blocking the movement of a needle directed towards the tube.

In one embodiment, the shielding device comprises a plurality of individual shields. Each individual shield may have a bell-like shape, a cone-like shape, a cylindrical shape, a bullet-like shape, or a ball and socket shape. The individual shields are positioned adjacent to each other along the tube. Each individual shield may be independently moveable to allow the tube to bend. Portions of adjacent individual shields overlap each other to assure no portion of the tube is exposed to an incoming needle. In addition, multiple different shapes of individual shields may be alternatively placed along the tube.

In one embodiment, the shielding device comprises a coil wrapped around the outer circumference of the tube. The coil is wrapped such that no portion of the tube is exposed to the needle. The coil may include a single wire, or multiple wires wrapped around the tube. In addition, multiple layers of wire may be wrapped over each other around the tube to further assure a needle cannot puncture the tube. Furthermore, the coil may have a size that is small enough to be integrated within the tube, as an alternative to placing it around the tube. The coil may be made from metal or a hard plastic or polymer.

In one embodiment, the shielding device has a flattened disk-like shape and is coupled to the access port. The flattened disk extends outward from the access port in a radial dimension to cover a portion of the tube. The shielding device may comprise multiple flattened disks extending outward from the access port, or a half-disk shape extending from the access port in a direction towards the tube. In addition, the shielding device may have multiple layers of material pressed together or sandwiched together to increase puncture resistance. The flattened disk may be a flexible disk, made from a flexible puncture resistant fabric or a hard material such as plastic.

The present invention includes a shielding device for shielding from and preventing needle puncture to a tubing for the conduct of a fluid, the tubing extending between and for providing bi-directional passage of a fluid between a subcutaneously implantable access port and a hydraulically inflatable portion of a gastric band, the gastric band intended for circumscribing the stomach of an obese patient. In one embodiment, the shielding device includes a substantially spiral-shaped body portion. The spiral-shaped body portion forms wraps around the tube. An extended portion of the shielding device extends from the body portion, and covers the portion of the tube positioned between adjacent wraps of the body portion. The body portion and/or the extended portion may be made of a puncture resistant material or materials.

In another embodiment of a shielding device for use to protect a tube from puncture, the tube extending from an access port of a gastric band system for the treatment of obesity, the shielding device can comprise a plurality of arcuate or curved and overlapping parallel ribs made of a puncture resistant material, the ribs extending around substantially all the circumference of the tube adjacent the access port. The ribs can extend around about 270 degrees of the circumference of the tube and the shielding device can be made by an over-molding process.

### BRIEF DESCRIPTION OF THE DRAWINGS

FIG. 1 illustrates a gastric band system according to an embodiment of the present invention.

FIG. 2 illustrates a perspective view of the inner diameter of the band corresponding to a decreased volume of fluid in the gastric band according to an embodiment of the present invention.

FIG. 3 illustrates a perspective view of the inner diameter of the band corresponding to an increased volume of fluid in the gastric band according to an embodiment of the present invention.

FIG. 4 illustrates a perspective view of the gastric band system removed from an individual's body according to an embodiment of the present invention.

FIG. 5 illustrates a side, cut-away view of the access port attached to the muscle wall of an individual according to an embodiment of the present invention.

FIG. 6 illustrates a perspective, close-up view of the shielding device and the access port according to an embodiment of the present invention.

FIG. 7 illustrates a side, cut-away view of the shielding device in operation according to an embodiment of the present invention.

FIG. 8 illustrates a side, cross-sectional view of the shielding device according to an embodiment of the present invention.

FIG. 9 illustrates a side, cross-sectional view of the shielding device according to an embodiment of the present invention.

FIG. 10 illustrates a side, cross-sectional view of the shielding device according to an embodiment of the present invention.

FIG. 11 illustrates a side, cross-sectional view of the shielding device according to an embodiment of the present invention.

FIG. 12 illustrates a side, cross-sectional view of the shielding device according to an embodiment of the present invention.

FIG. 13 illustrates a side, cross-sectional view of the shielding device according to an embodiment of the present invention.

FIG. 14 illustrates a side view of the shielding device according to an embodiment of the present invention.

FIG. 15 illustrates a side, close-up view of the shielding device according to an embodiment of the present invention.

FIG. 16 illustrates a side, cross-sectional view of the shielding device according to an embodiment of the present invention.

FIG. 17 illustrates a perspective view of the shielding device according to an embodiment of the present invention.

FIG. 18 illustrates a top view of the shielding device according to an embodiment of the present invention.

FIG. 19 illustrates a side, cut-away view of the shielding device in operation according to an embodiment of the present invention.

FIG. 20 illustrates a top view of the shielding device according to an embodiment of the present invention.

FIG. 21 illustrates a side, cut-away view of the shielding device in operation according to an embodiment of the present invention.

FIG. 22 illustrates a side, cross-sectional view of the shielding device according to an embodiment of the present invention.

FIG. 23 illustrates a side, cross-sectional view of the shielding device according to an embodiment of the present invention.

FIG. 24 illustrates a side, cross-sectional view of the shielding device according to an embodiment of the present invention.

FIG. 25 illustrates a side, cross-sectional view of the shielding device according to an embodiment of the present invention.

FIG. 26 illustrates a perspective view of a shielding device according to an embodiment of the present invention.

FIG. 27 illustrates a perspective view of a shielding device according to an embodiment of the present invention.

FIG. 28 illustrates a side, cross-sectional view of a shielding device according to an embodiment of the present invention.

FIG. 29 illustrates a side, cross-sectional view of a shielding device according to an embodiment of the present invention.

FIG. 30 illustrates a side, cross-sectional view of a shielding device according to an embodiment of the present invention.

FIG. 31 illustrates a side view of the shielding device according to an embodiment of the present invention.

FIG. 32 illustrates a side, cross-sectional view of a shielding device according to an embodiment of the present invention.

FIG. 33 is a front elevation view of a prior art injection port with tubing and strain relief.

FIGS. 34A and 34B illustrate two tubing protector embodiments.

FIG. 35 is an exploded, elevation view showing an over molding assembly for making the tubing protector of FIG. 34A.

FIG. 36 illustrates the injection port, tubing and strain relief of FIG. 33 with the FIG. 34A tubing protector (made in the over molding assembly of FIG. 35) in place.

FIG. 37 illustrates a further embodiment of a tubing protector.

### DETAILED DESCRIPTION

The present invention relates to a shielding device that protects a tube or tubing used in a gastric band system. Specifically, the shielding device protects a tube or tubing from puncture by a syringe needle inserted near the tube or tubing.

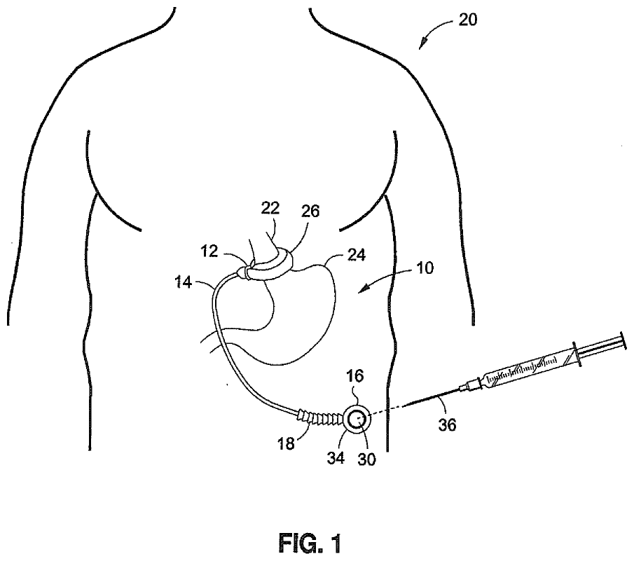

As shown in FIG. 1, the gastric band system 10 includes a band 12 (e.g., a gastric band 12), a tube 14, an access port 16, and a shielding device 18 placed over a portion of the tube 14. The gastric band system 10 is surgically implanted within an individual's body 20. A physician places the band 12 around the upper portion 22 of an individual's stomach 24 and fixes the access port 16 to a portion of the individual's body 20. Preferably, the access port 16 is securely fixed to the muscle wall of the abdomen inside the individual's body 20. The tube 14 connects the band 12 to the access port 16. The shielding device 18 is positioned completely around the tube 14, adjacent to the access port 16.

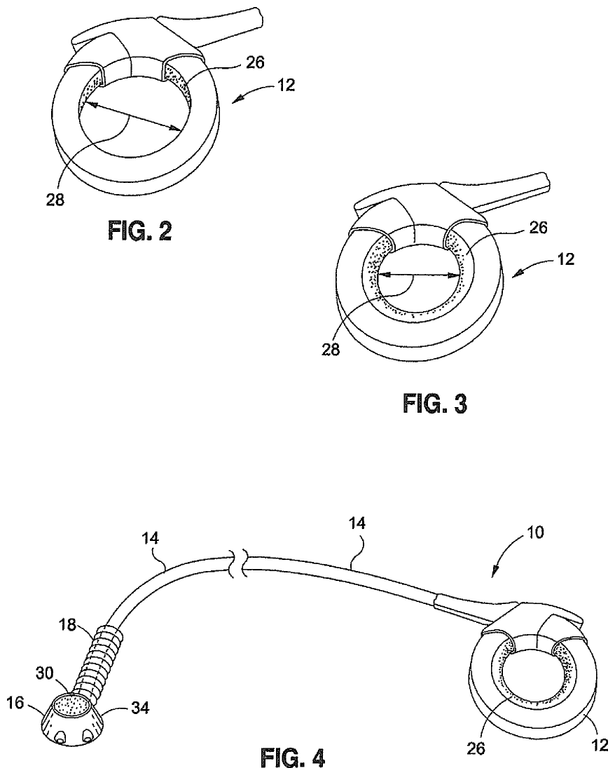

The gastric band system 10 shown in FIG. 1 operates in response to a hydraulic force. The band 12 includes an inner bladder 26 defining an inner diameter 28 (shown in FIG. 2) with a size that varies based on the volume of fluid inside the inner bladder 26. The volume of fluid inside the inner bladder 26 may be controlled by a physician through the access port 16. The access port 16 may include a septum 30, a fluid chamber 32 (shown in FIG. 8), and an access port housing 34 holding the fluid chamber 32 and the septum 30. The septum 30 is configured as a membrane located over the fluid chamber 32, to allow a syringe needle 36 to pass through the septum 30 and into the fluid chamber 32 to deposit or remove fluid. The septum 30 is preferably made from a soft needle-penetrable material such as silicone. The tube 14 has two ends, with one end coupled to the fluid chamber 32 and one end coupled to the inner bladder 26 of the band 12. The tube 14 transfers the fluid from the fluid chamber 32 to the inner bladder 26 of the band 12. In this configuration, a physician can control the size of the inner bladder 26 by inserting a syringe needle 36, or long hypodermic needle, through the surface of the individual's skin, through the septum 30, and into the fluid chamber 32, to either deposit or inject fluid into or remove fluid from the gastric band 12.

If the physician deposits or injects fluid into the fluid chamber 32, the inner bladder's 26 inner diameter 28 decreases, and the band 12 constricts the upper portion 22 of the stomach 24. The constricted upper portion 22 of the stomach 24 reduces the flow of food passing to the lower part of the stomach 24, ideally causing the individual to lose weight over time. If the physician removes fluid from the fluid chamber 32, the inner bladder's 26 inner diameter 28 increases, and band 12 loosens around the upper portion 22 of the stomach 24. The flow of food passing to the lower part of the stomach 24 correspondingly increases.

FIG. 2 illustrates an increased size of the inner diameter 28 corresponding to a decreased volume of fluid in the inner bladder 26 of the gastric band 12.

FIG. 3 illustrates a decreased size of the inner diameter 28 corresponding to an increased volume of fluid in the inner bladder 26 of the gastric band 12.

FIG. 4 illustrates a perspective view of the gastric band system 10 when it is not installed within the interior of the individual's body 20.

To adjust the size of the inner bladder 26, the physician may need to repeatedly insert a syringe needle 36 into the individual's body 20 to add or remove fluid from the gastric band system 10. Also, the physician may need to insert a syringe needle 36 on a periodic basis to adjust the size of the inner bladder 26, or to assure the fluid pressure is sufficient in the gastric band system 10. As such, it is important that the physician be able to easily identify and locate the precise position of the septum 30.

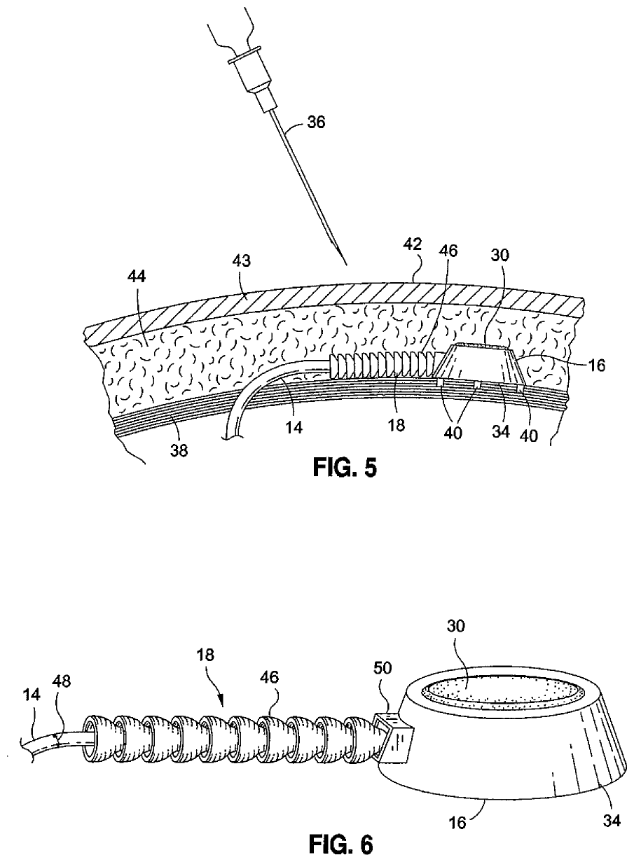

FIG. 5 shows a side, cut-away view of the access port 16 attached to or engaged with the abdominal muscle wall 38 of the individual. As discussed above, a physician may surgically implant the access port 16 to the muscle wall 38 of an individual. The muscle wall 38 provides a secure attachment point to assure the access port 16 does not travel throughout the individual's body 20 and potentially disengage from the tube 14. The access port 16 is configured to attach to bodily tissue. A plurality of anchors 40 may be used to fix the access port 16 to the muscle wall 38. These anchors 40 may comprise hooks or barbs that penetrate the muscle wall 38 and fix the access port 16 in place.

When the physician attaches the access port 16 to the muscle wall 38, the physician also passes the tube 14 inside the individual's body 20 to connect to the inner bladder 26. The tube 14 should remains flexible to allow the physician to easily manipulate the tube 14 during insertion. Accordingly, the tube 14 may be made of a durable, flexible material such as silicone or other equivalent material.

A drawback to fixing the access port 16 to the muscle wall 38 is that the position of the septum 30 may change over time relative to the surface 42 of the skin 43. The amount of fat 44 located around the access port 16 may vary, shifting the position of the access port 16 relative to the surface 42 of the skin 43. In this situation, the physician may not be able to detect the exact position of the septum 30. Therefore, it may be difficult for the physician to repeatedly determine the exact position of the septum 30 over an extended period of time, if the patient's weight is changing. A physician can place a mark on the skin 43 to indicate the position of the septum 30; however, the mark may deviate from the septum 30 over time. To properly locate the septum 30, the physician can also palpate the area around the access port 16 to generally feel where the septum 30 is located. However, even a skilled physician may not correctly determine the precise location of the septum 30 because it may be under many layers of fat 44.

The physician may therefore incorrectly insert the syringe needle 36 through the skin 43 and contact the muscle wall 38. Although this result may be painful, another problem may occur if the syringe needle 36 penetrated the tube 14. As discussed above, the tube 14 is typically made from a soft, flexible material such as silicone, which may be easily penetrated by a syringe needle 36. If the tube 14 is punctured, the pressurized fluid in the tube 14 may leak out into the individual's body 20. The gastric band system 10 would then be inoperable, and the physician would either need to surgically remove the gastric band system 10 or perform an operation to mend the punctured tube 14. To alleviate the problem of a punctured tube 14, the shielding device 18 may be placed over a portion of the tube 14 located adjacent to the access port 16. In one embodiment, the shielding device 18 is placed completely around the tube 14 so that the tube 14 is protected from all sides and directions.

FIG. 6 displays a perspective view of one embodiment of the shielding device 18. The shielding device 18 may comprise a plurality of individual shields 46, or beads, coupled to the tube 14 and spaced adjacent to one another. Each individual shield 46 has a generally cylindrical shape that entirely wraps around an outer circumference 48 of the tube 14. Each individual shield 46 may be made from a hard, puncture resistant material that is impenetrable by the needle 36 inserted by the physician. The material may be a hard plastic, a light-weight metal, a ceramic, or a hardened polymer, or a thermoplastic such as polysulfone. Generally, the material is hard enough that the syringe needle 36 is incapable of piercing the puncture resistant material, beyond merely placing a small divot or scratch on the surface of the material. The shielding device 18 covers the end of the tube 14 and is positioned close enough to the access port 16 to block a misplaced needle 36 inserted by the physician. For example, the shielding device 18 may be attached to and positioned adjacent to the access port housing 34 such that no gap exists between the shielding device 18 and the access port housing 34. In addition, the access port housing 34 may include a protective canopy structure 50 to assure a needle 36 traveling towards the tube 14 cannot contact an area of exposed tube 14 between the shielding device 18 and the access port housing 34. The shielding device 18 protects the tubing from needle sticks while remaining flexible and provides strain relief for the tubing.

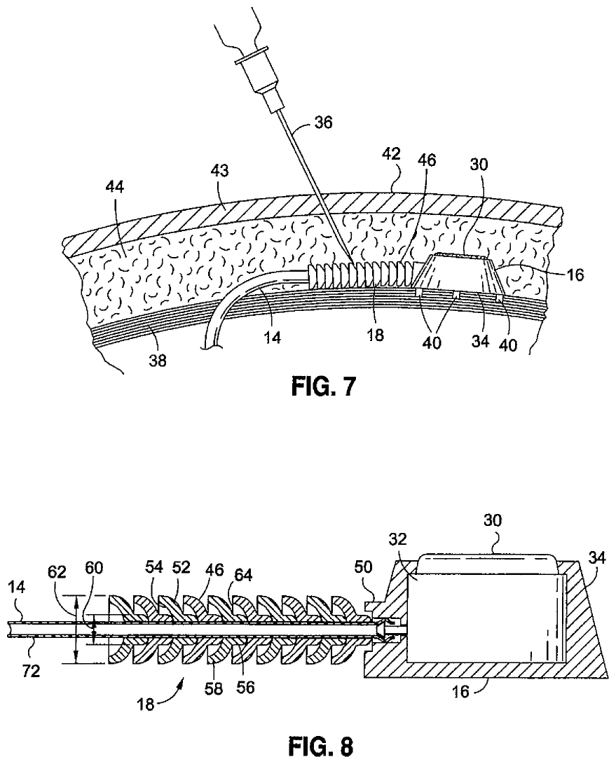

The operation of the shielding device 18 is shown in FIG. 7. When a physician inserts the needle 36, the shielding device 18 blocks the motion of the needle 36 and prevents it from penetrating the tube 14. Because the shielding device 18 is made from a hard material, the physician may feel the syringe needle 36 hit a hard surface and will know the needle 36 is not contacting the septum 30. The physician may then retract the syringe and attempt to find the septum 30 again. The tube 14 will be protected from puncture.

In an alternative operation, the shielding device 18 may be composed of a puncture resistant material that merely resists penetration by a needle 36. The puncture resistant material may deform when contacted by the needle 36, but the energy required to pass through the shielding device 18 and contact the tube 14 may be great. The physician will notice the increased resistance and realize the needle 36 is not contacting the septum 30.

FIG. 8 illustrates a cross-section view of the shielding device 18 showing the shape and position of each individual shield 46 along the tube 14. In this embodiment, each individual shield 46 has a generally bell-like shape, with a curved outer surface 52 and curved inner surface 54. Each individual shield 46 has a neck portion 56 and an extended portion 58. Both the neck portion 56 and extended portion 58 have an associated diameter, with the diameter 60 of the neck portion 56 being smaller than the diameter 62 of the extended portion 58. The different diameters 60, 62 allow the extended portion 58 to form a hollow cavity 64 defining the inner surface 54. Thus, the extended portion 58 defines the hollow cavity 64 for receiving the neck portion 56 from an adjacent shield 46. The neck portion 56 of an adjacent individual shield 46 may enter into a portion of the hollow cavity 64. The neck portion 56 and the extended portion 58 of the adjacent individual shields 46 therefore overlap slightly and are moveably connected to one another. The curved shape of the outer surface 52 and the inner surface 54 allow the neck portion 56 to more easily enter the hollow cavity 64. The neck portion 56 of an individual shield 46 enters into the hollow cavity 64 to assure a syringe needle 36 cannot directly contact the tube 14 if it is inserted in a perpendicular direction towards the tube 14. If the extended portion 58 does not extend over the neck portion 56 of the adjacent individual shield 46, a small gap of exposed tube 14 may exist between the individual shields 46. The needle 36 can then penetrate the tube 14 at the exposed areas.

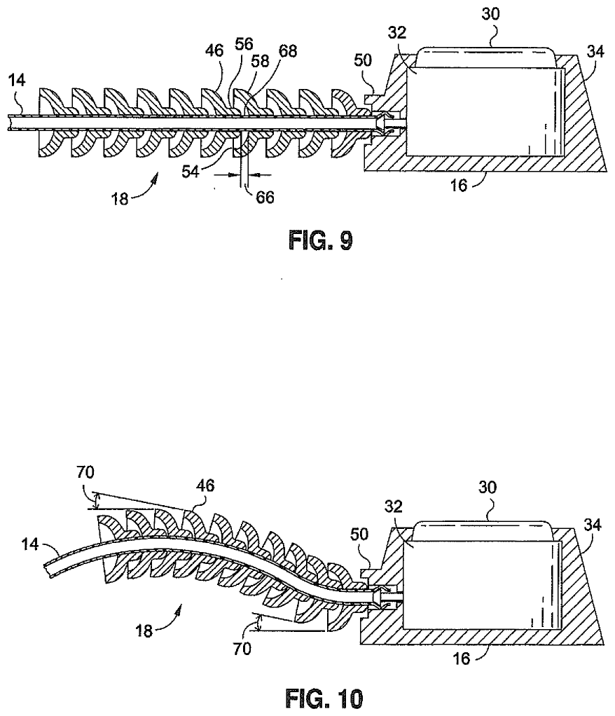

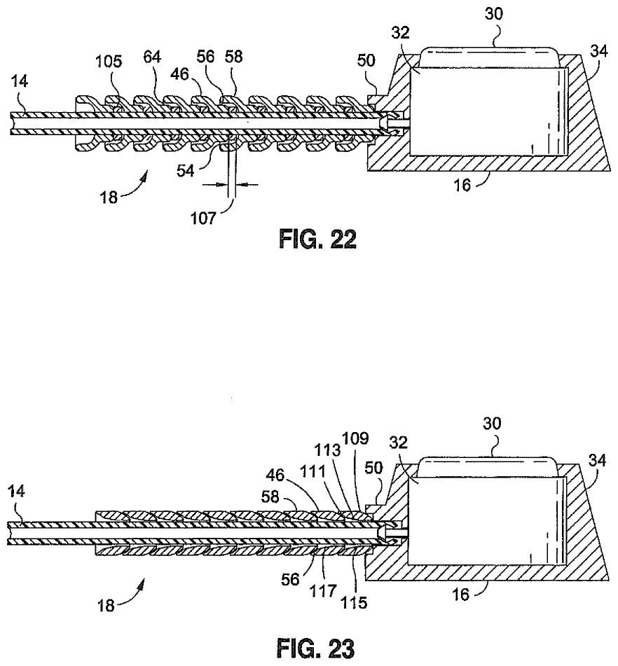

The individual shields 46 are spaced along the tube 14 equidistantly, at regular intervals from each other. However, the spacing between the individual shields 46 may vary in different embodiments. In the embodiment shown in FIG. 8, each individual shield 46 is spaced such that the neck portion 56 contacts or very nearly contacts the inner surface 54 of an adjacent individual shield 46. In this configuration, no gap exists between the adjacent individual shields 46. However, in the embodiment shown in FIG. 9, the individual shields 46 may be spaced such that a small gap 66 exists between the neck portion 56 of an individual shield 46 and the inner surface 54 of an adjacent individual shield 46. The gap 66 increases the flexibility of the portion of the tube 14 protected by the shielding device 18. The gap 66 may be formed by gluing the individual shields 46 at a distance from each other, or spacers may be used, as discussed in relation to FIG. 22. As discussed above, it may be beneficial to have the tube 14 be flexible during insertion into an individual 20. A size or shape of the extended portion 58 of an individual shield 46 may be modified to assure the exposed tube portion 68 between the individual shields 46 is still protected from an incoming needle 36.

FIG. 10 illustrates the flexibility of the shielding device 18 for the embodiment shown in FIG. 8. Each individual shield 46 may rotate with respect to the position of an adjacent individual shield 46. The angle of rotation 70 may be based on a plurality of factors, including the length and shape of the extended portion 58, the distance of the individual shields 46 to each other, and the overall flexibility of the material comprising the tube 14 and the individual shields 46. The flexibility of the shielding device 18 is an advantage over an embodiment simply including a hard metal or plastic sheath placed over a portion of the tube 14. A hard sheath placed over a portion of the tube 14 would not allow a physician to easily manipulate the tube 14 when inserted into an individual 20. The plurality of individual shields 46 allow a hard, inflexible, material to be attached to the tube 14, yet allow the tube 14 to remain flexible for easy manipulation. In addition, a flexible tube 14 is also important for patient comfort. For example, if the patient were to bend over, a rigid shielding device may exert more pressure on the surrounding tissues than a flexible one, resulting in pain.

Referring back to FIG. 8, each individual shield 46 may be individually coupled to the outer surface 72 of the tube 14. In one embodiment, the individual shields 46 are not directly coupled to each other but rather coupled to the outer surface 72 of the tube 14. The individual shields 46 may be slid onto the tube 14 and then fixed in place along the tube 14 with silicone glue or other equivalent attachment means. An individual shield 46 may therefore not slide along the tube 14 or move laterally relative to another individual shield 46. The individual shields 46 may be immovably fixed to the tube 14. In addition, if the individual shields 46 are coupled directly to the tube 14, the access port housing 34 does not need to be modified. The tube 14 may be disengaged from the access port housing 34, and the shielding device 18 will remain attached to the tube 14.

However, in one embodiment, the individual shields 46 may be fixed to the tube 14 in another manner. For example, each individual shield 46 may be fixed to a flexible sleeve (not shown), and the flexible sleeve may be slid over the tube 14. The flexible sleeve may be directly attached to the access port housing 34 or glued to the outer surface 72 of the tube 14. The flexible sleeve may allow the shielding device 18 to be entirely disengaged from the tube 14 and the access port housing 34 during assembly or disassembly of the gastric band system 10.

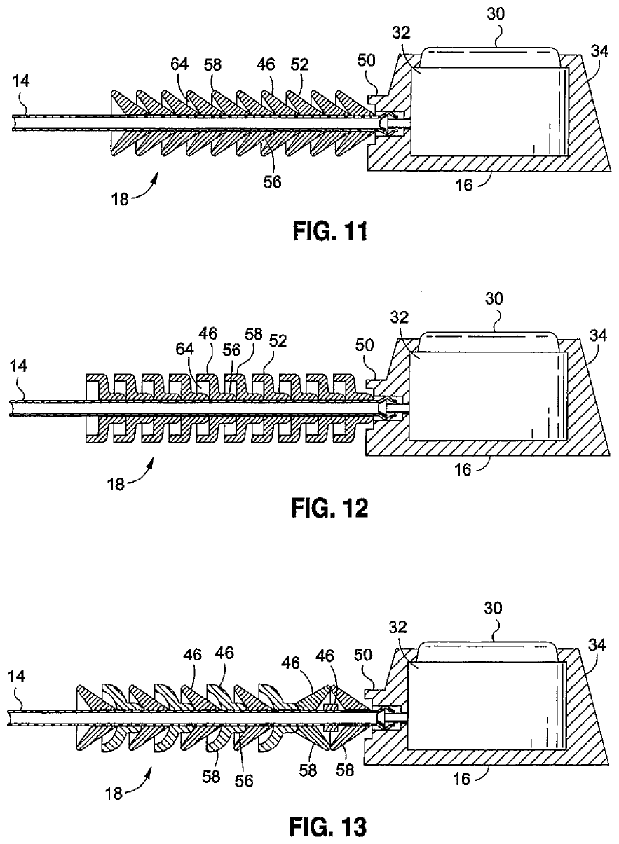

FIG. 11 illustrates a cross-sectional view of an embodiment of the shielding device 18 with each individual shield 46 having a generally cone-like shape. Similar to the embodiment shown in FIG. 8, each individual shield 46 has a neck portion 56 and an extended portion 58. However, in this embodiment, the outer surface 52 of the individual shield 46 has a flattened shape, and the hollow cavity 64 has a conical shape. The neck portion 56 of the individual shield 46 extends into the extended portion 58 of an adjacent individual shield 46. Similar to the embodiment shown in FIG. 8, the overlap of the extended portion 58 over the neck portion 56 protects the tube 14 from contact with an incoming syringe needle 36. In addition, similar to the embodiment shown in FIG. 8, the size of an extended portion 58 and the distance between adjacent individual shields 46 may be varied to offer different levels of flexibility and protection for the tube 14.

FIG. 12 illustrates a cross-section view of an embodiment of the shielding device 18 with each individual shield 46 having a more cylindrical shape than the embodiment shown in FIG. 8. Similar to the embodiment shown in FIG. 8, each individual shield 46 has a neck portion 56 and an extended portion 58. However, in this embodiment, the outer surface 52 of the individual shield 46 has a more flattened shape, and the hollow cavity 64 has a cylindrical shape. The neck portion 56 of the individual shield 46 extends into the extended portion 58 of an adjacent individual shield 46. Similar to the embodiment shown in FIG. 8, the overlap of the extended portion 58 over the neck portion 56 protects the tube 14 from contact with an incoming syringe needle 36. In addition, similar to the embodiment shown in FIG. 8, the size of an extended portion 58 and the distance between adjacent individual shields 46 may be varied to offer different levels of flexibility and protection for the tube 14.

FIG. 13 illustrates one embodiment of the shielding device 18 utilizing a combination of cone-shaped individual shields 46 and bell-shaped individual shields 46. The cone-shaped individual shields 46 and bell-shaped individual shields 46 may be alternatively placed along the length of the tube 14. In addition, similarly shaped individual shields 46 may be placed in a different orientation with respect to one another. For example, a cone-shaped individual shield 46 may have an extended portion 58 directed towards an extended portion 58 of an adjacent cone-shaped individual shield 46. The embodiment shown in FIG. 13 also illustrates an individual shield 46 may have no defined extended portion 58 or neck portion 56. As shown in FIG. 13, the shape, orientation, and position of the individual shields 46 may be varied to produce alternative degrees of flexibility and protection for the tube 14.

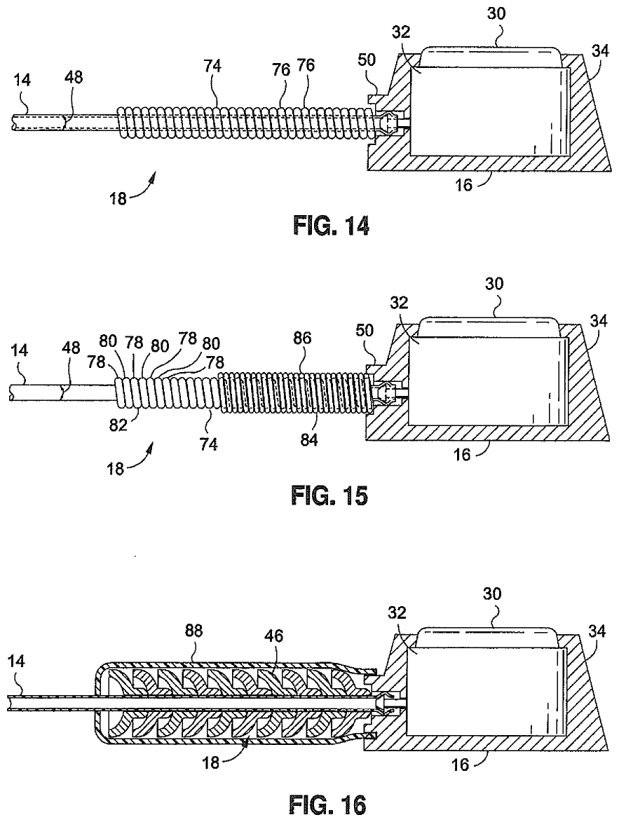

FIG. 14 illustrates an embodiment of the shielding device 18 utilizing a wire or hard tubing wrapped multiple times over a portion of the tube 14, forming a coil 74. The coil 74 encircles the exterior circumference 48 of the tube 14. The coil 74 may be comprised of a hard material, such as a metal wire, or a flexible hard plastic or polymer. The metal may comprise titanium, nitinol, other non-ferrous relatively flexible materials, or a similar biocompatible metal.

The coil 74 is positioned adjacent to the access port housing 34, to leave no gap between the coil 74 and the access port housing 34 for a syringe needle 36 to contact the tube 14. In addition, the tightly wound wraps 76 of the coil 74 are spaced closely, and may contact each other, to leave no gap for a syringe needle 36 to pass through the shielding device 18 and contact the tube 14.

The multiple wraps 76 of the coil 74 allow the shielding device 18 to remain flexible, yet still be comprised from a hard material. A wrap 76 of the coil 74 may rotate relative to an adjacent wrap 76 of the coil 74. The coil 74 may be fixed to the tube 14 directly, through a silicone glue or equivalent means of fixing the coil 74. In addition, a portion of the coil 74 may be coupled directly to the access port housing 34, to further secure the coil 74 in place along the tube 14.

FIG. 15 illustrates an embodiment of the shielding device 18 shown in FIG. 14 utilizing two different wires 78, 80 wrapped around the tube 14 to form an inner coil 82. A secondary or outer coil 84 is also placed over and around the inner coil 82. The secondary coil 84 is wrapped multiple times around an exterior circumference of the inner coil 82. The two different wires 78, 80 may be wrapped alternatively around the tube 14. The wraps may be spaced near each other or in direct contact with each other. It is beneficial to utilize two wires 78, 80 if, for example, one of the wires 78, 80 breaks. The other wire may hold the coil 74 in place around the tube 14. In addition, each wire 78, 80 may be composed of a different material. One wire may be made from a more flexible material and one wire may be made from a material that is harder but less flexible. The different materials may provide a varying amount of flexibility and strength for the coil 74.

The secondary coil 84 comprises a wire 86 wrapped over the surface of the inner coil 82. The wire 86 of the secondary coil 84 includes wraps positioned close to or in contact with each other. The wire 86 of the secondary coil 84 may have a narrower diameter than a wire 78, 80 of the inner coil 82 to allow the secondary coil 86 to more easily flex when the tube 14 is manipulated. The secondary coil 84 may be placed along the entire length of the inner coil 82 or over a portion of the inner coil 82 adjacent to the access port housing 34. Although FIG. 15 illustrates three wires 78, 80, 86 wrapped around the exterior circumference 48 of the tube 14, many more layers or many more wires may be used to form a coil 74 around the tube 14.

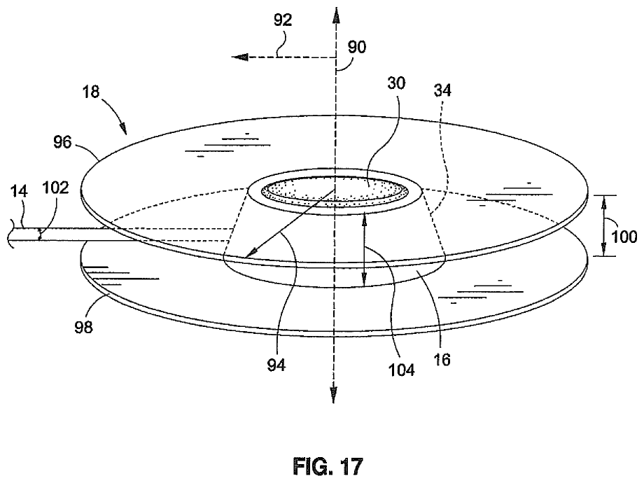

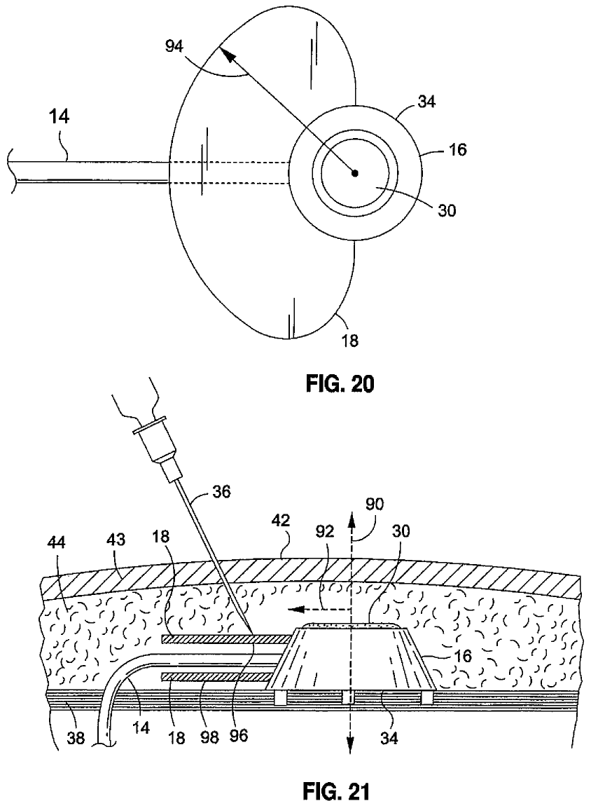

FIG. 16 illustrates an embodiment of the shielding device 18 including a cylindrical sheath 88 placed over the entirety of the shielding device 18. The cylindrical sheath 88 may comprise an overmolding of silicone placed over the shielding device 18. The silicone overmolding may provide a greater degree of biocompatibility for the shielding device 18 and provides further strain relief for the tube 14. In addition, the cylindrical sheath 88 may smooth the surface of the shielding device 18 to allow the tube 14 to be more easily inserted into an individual's body 20. The cylindrical sheath 88 may be combined with any of the embodiments discussed herein, including the embodiments shown in FIGS. 17 and 20.

FIG. 17 illustrates an embodiment of the shielding device 18 having a flattened disk-like or skirt-like shape. In this configuration, the shielding device 18 is fixed directly to the access port housing 34. The access port housing 34 may define a radial dimension 92 and an axial dimension 90. The shielding device 18 extends from the access port housing 34 in a radial direction, and in the radial dimension 92, away from the access port housing 34. The shielding device 18 covers the end of the tube 14 from a syringe needle 36 traveling towards the tube 14. The size of the radius 94, or distance from the access port 16, formed by the shielding device 18 determines the extent of the tube 14 covered by the shielding device 18. In one embodiment, the size of the radius 94 may be greater than twice a diameter of the access port 16. In the embodiment shown in FIG. 17, the shielding device 18 may include two disks, a top disk 96 and a bottom disk 98. The end of the tube 14 passes between the two disks 96, 98. The distance 100 between the top disk 96 and bottom disk 98 may define the flexibility of the tube 14 and the amount of protection for the tube 14. For example, if the two disks 96, 98 are placed relatively near each other (e.g., spaced at the diameter 102 of tube 14), then the tube 14 may be trapped between the two disks 96, 98 and cannot move too much. However, the disks 96, 98 protect the tube 14 from a needle 36 passing towards the tube 14 at a relatively horizontal angle relative to the access port housing 34. If the disks 96, 98 are placed relatively far from each other (e.g., spaced at the height 104 of the access port housing 34), the tube 14 may be more flexibly manipulated, but the disks 96, 98 offer less protection from the needle 36 being able to pass toward the tube 14 horizontally. The shielding device 18 may also comprise a single top disk 96 placed above the tube 14 to protect the tube from a needle 36 traveling in an axial direction.

The disk-like or skirt-like shaped shielding device 18 allows the tube 14 to be shielded without any attachment or modification to the tube 14, unlike the embodiment shown in FIG. 8. The tube 14 retains its flexibility, only limited by the dimensions of the shielding device 18, as discussed above. However in this embodiment, the access port housing 34 is modified. The shielding device 18 may be firmly fixed to the access port housing 34 or removably fixed to the access port housing 34. If the shielding device 18 is removably fixed, it may be snap-fit to an outer portion of the access port 16. The shielding device 18 may be made out of a puncture resistant material, including a hard plastic, metal, ceramic, or hard polymer. In addition, the shielding device 18 may be made from a fabric material such as several layers of a tightly woven nylon or polyester, woven quartz or silica fibers, or the equivalent. The fabric material provides puncture resistance, but also allows the shielding device 18 to flex or bend to conform to the patient's body, or allow for easy insertion into the patient's body. Thus, the shielding device 18 may comprise a flexible disk-like or skirt-like shaped device. In addition, each disk 96, 98 may comprise a single layer of a puncture resistant material, or multiple layers of a puncture resistant material compressed or sandwiched together.

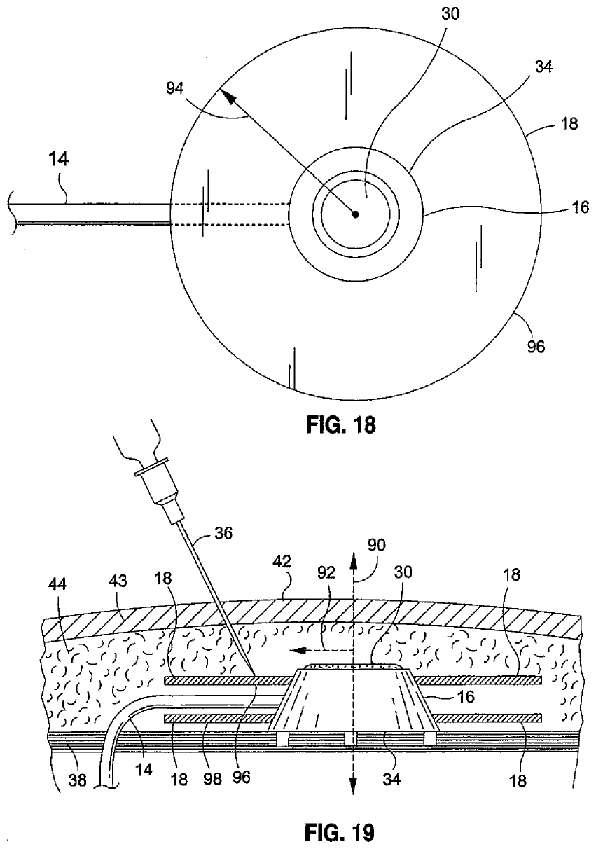

FIG. 18 illustrates a top view of the shielding device 18 as shown in FIG. 17. The top view illustrates the shielding device 18 extending out radially from the access port 16 and covering a portion of the tube 14. The shielding device 18 extends radially around the entirety of the access port 16, or, in other words, 360 degrees around the axis of the axial dimension 90 shown in FIG. 17.

FIG. 19 illustrates the shielding device 18 in operation. Similar to the operation of the shielding device 18 shown in FIG. 8, if a physician incorrectly inserts a syringe needle 36 towards the tube 14, the needle 36 may contact the shielding device 18. The physician may notice the syringe needle 36 has contacted a hard material, and will know the needle 36 did not contact the septum 30. The tube 14 will not be punctured.

FIG. 20 illustrates a top-view of an alternate shape of the shielding device 18 shown in FIG. 17. In this embodiment, the shielding device 18 may have a disk-like shape that does not extend radially around the entirety of the access port housing 34. The shielding device 18 only extends radially in a direction (i.e., one direction) towards the tube 14, and only extends radially around a portion of the access port 16 (e.g., half of the access port housing 34, or 180 degrees around axis of the axial dimension 90 shown in FIG. 17). The modified disk shape, or half-disk shape, may offer less protection for the tube 14 around the entire access port housing 34. However, the half-disk shape also provides the access port housing 34 with a smaller total size. The smaller size may make it easier for a physician to insert the access port 16 into an individual's body.

FIG. 21 illustrates the shielding device 18 shown in FIG. 20 in operation. The shielding device 18 blocks a syringe needle 36 from contacting the tube 14. The shielding device 18 in this embodiment only extends around a portion of the access port housing 34 in a direction towards the tube 14.

FIG. 22 illustrates an embodiment of the shielding device 18 including spacers 105 that have an annular shape, placed between the individual shields 46. The spacers 105 may extend entirely around the outer surface of the tube 14 and may be positioned between the individual shields 46. The spacers 105 may be positioned within the hollow cavity 64 that is defined by the extended portion 58. A width 107 of the spacer 105 may be used to define a distance between the individual shields 46. The spacers 105 may be made of a pliable material, such that the spacers 105 may compress when the individual shields 46 are rotated with respect to each other. Such pliable material may include a soft plastic or the like. In addition, the spacers 105 may also be made of a hard material, but may be sized small enough to still allow the individual shields 46 to rotate. The spacers 105 may have a variety of shapes, including, but not limited to an o-ring shape, a tubular shape, or a toroid shape. The spacers 105 are used to space the shields 46 from each other. In addition, the spacers 105 may also provide protection for the tube 14, and may be made from a needle impenetrable material. The spacer 105 may be designed to protect the exposed areas of the tube 14 positioned between the individual shields 46. The spacers 105 may be firmly fixed to the tube 14 in any manner discussed previously in this application.

FIG. 23 illustrates an embodiment of the shielding device 18 including bullet-like shaped individual shields 46. In this embodiment, each individual shield 46 has an external articulating surface 109, an internal articulating surface 111, a cylindrical surface 115, and a conical surface 113. The portion of the shield 46 near the cylindrical surface 115 generally comprises the neck portion 56 of the shield 46. The portion of the shield 46 positioned near the internal articulating surface 111 generally comprises the extended portion 58 of the individual shield 46. In this embodiment, the internal articulating surface 111 extends over the external articulating surface 109 of an adjacent shield. In this manner, the two surfaces 111, 109 form a congruent fit around the circumference of the tube 14. The two surfaces 111, 109 may contact each other, to assure a syringe needle cannot penetrate through a gap in the shielding device 18. The two surfaces 111, 109 may have a corresponding arc shapes, or curved shapes, that may allow them to contact each other with a substantial amount of surface area.

The cylindrical surface 115 is shaped to wrap around the tube 14, and may grip the tube or may be glued directly to the tube 14. In addition, the cylindrical surface 115 may be slightly larger than the tube 14. The shielding device 18 in this embodiment remains flexible, in part, because of the conical surface 113 positioned between the internal articulating surface 111 and the cylindrical surface 115. A portion of the conical surface 113 may be shaped to extend in a direction away from the surface of the tube 14 with a generally conical shape. One end of the conical surface 113 is positioned near the tube 14 and another end extends away from the tube 14. The end of the conical surface 113 positioned away from the tube 14 transitions to the internal articulating surface 111, which, as discussed above, has a curved shape to conform to a curved or arc shape of the external articulating surface 109.

The shape of the conical surface 113 forms an interior cavity 117 positioned between the tube 14 and the individual shield 46. The interior cavity 117 allows the individual shield 46 to rotate, or articulate around the tube 14 when the tube 14 is flexed. No portion of an adjacent individual shield 46 extends into the interior cavity 117.

When the tube 14 is flexed, the internal articulating surface 111 and the external articulating surface 109 slide with respect to one another and compress or expand a portion of the interior cavity 117. The arc shape of the surfaces 111, 109 aids the sliding motion of the shields 46. In addition, when the tube 14 is flexed, one portion of the external articulating surface 109 slides away from the respective portion of the internal articulating surface 111, and a portion of the external articulating surface 109 slides towards the respective portion of the internal articulating surface 111 simultaneously. The two portions of the external articulating surface 109 may be positioned opposite from one another around the individual shield 46. The external articulating surface 109 and the internal articulating surface 111 remain in contact, or remain close to one another when the tube 14 is flexed. This configuration allows for a closely guarded, yet flexible tube 14. The design eliminates the need for spacers between the shields 46 and minimizes any gaps between the shields 46. The sizes or particular shapes of the individual shields 46 in this embodiment may be varied to produce alternative, equivalent results. The individual shields 46 may be firmly fixed to the tube 14 in any manner discussed previously in this application.

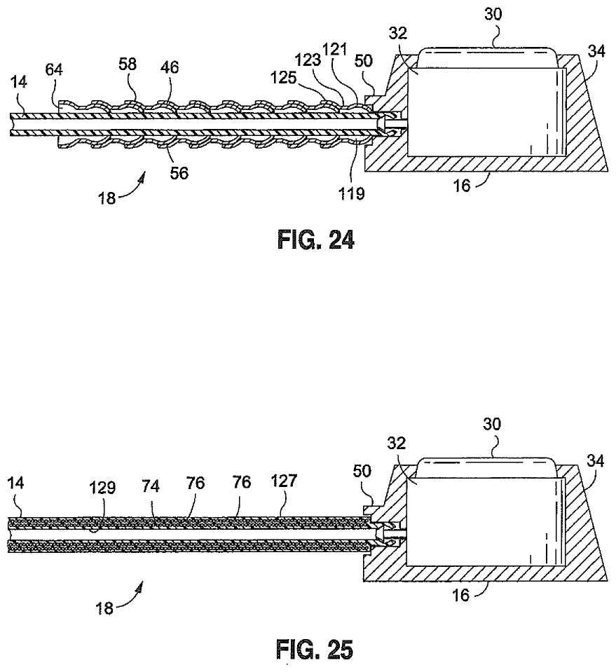

FIG. 24 illustrates an embodiment of the shielding device 18 including ball and socket shaped individual shields 46. In this embodiment, each individual shield 46 has an external spherical surface 121, a narrow portion 123, and a spherical housing portion 125. The spherical housing portion 125 extends around the external spherical surface 121 and has a curved, spherical shape corresponding to a curved, spherical shape of the external spherical surface 121. Thus, the spherical housing portion 125 may contact or nearly contact the external spherical surface 121. The spherical shape of both the spherical housing portion 125 and the external spherical surface 121 allow the connection between the two components 125, 121 to serve as a ball joint, allowing the tube 14 to flex, or rotate substantially. Each individual shield 46 may rotate with respect to an adjacent individual shield 46, limited by the extent that the spherical housing portion 125 wraps around the external spherical surface 121. In other words, if the housing portion 125 wraps entirely around the external spherical surface 121, then no rotation will be possible. In this embodiment, the external spherical surface 121 comprises the neck portion 56 of the individual shield 46, and the spherical housing portion 125 comprises the extended portion 58.

The rotation of the spherical housing portion 125 is limited by the narrow portion 123, which is positioned between the external spherical surface 121 and the spherical housing portion 125. The narrow portion 123 serves as a transition point between the external spherical surface 121 and the housing portion 125. If the individual shield 46 rotates too far in one direction, a portion of the spherical housing portion 125 contacts the narrow portion 123, preventing further movement.

The individual shields 46 additionally remain flexible around the tube 14 because the ball and socket shape forms a ball cavity 119, within the interior of the individual shield 46. The ball cavity 119 provides an area of movement for the individual shield 46, similar to the internal cavity 117 shown in FIG. 23. Thus, portions of the ball cavity 119 may be variably distanced from the surface of the tube 14 during movement of the tube 14. The ball cavity 119 may be formed because the external spherical surface 121 may only contact the tube 14 at a narrow portion, or a ring portion of the external spherical surface 121. Thus, the ball cavity 119 extends outward from the surface of the tube 14. The ring portion may be firmly fixed to the tube 14 in any manner discussed previously in this application.

Similar to the embodiment shown in FIG. 23, this configuration allows for a closely guarded, yet flexible tube 14. The design eliminates a need for spacers between the shields 46 and minimizes any gaps between the shields 46. The sizes or particular shapes of the individual shields 46 in this embodiment may be varied to produce alternative, equivalent results.

FIG. 25 illustrates an embodiment of the shielding device 18 including a coil 74 wrapped around an interior surface 129 of the tube 14. This configuration is similar to the embodiment shown in FIG. 14, but in this embodiment, the coil 74 is positioned within the tube 14. In other words, the coil 74 is small enough to fit within an exterior surface 127 of the tube 14, yet is large enough to extend around an interior surface 129 of the tube 14. The multiple wraps 76 of the coil 74 entirely encircle the interior surface 129 or interior circumference of the tube 14. The benefit of this embodiment is to reduce the size of the shielding device 18 to equal, or nearly equal the diameter of the tube 14 without a shielding device 18 attached. The tube 14 including the coil 74 would then have an overall smaller cross section than the embodiment shown in FIG. 14. This may be advantageous to allow a physician to more easily insert the tube into a patient's body.

Although FIG. 25 illustrates the tube 14 sized larger than the tube shown in FIG. 14, the sizing is for illustrative purposes only. In this embodiment, the tube 14 may have an equal total diameter, or smaller total diameter than shown in FIG. 14. In addition, the coil 74 may extend along only a portion of the tube 14 or may extend along the entirety of the tube 14 (e.g., from one end near or touching the housing 34 to the other end near or touching the gastric band 12). In addition, similar to the embodiment shown in FIGS. 14 and 15, the coil 74 may include multiple wraps of wire, multiple layers of wire wraps, or multiple wires wrapped around the interior surface 129 of the tube 14. The coil 74 in this embodiment, similar to the embodiment shown in FIG. 14, may be made from a metal such as titanium, nitinol or a hard plastic. The coil 74 may be molded into the tube 14 or fixed to the interior surface 129 of the tube 14 through any manner discussed above in relation to FIGS. 14 and 15.

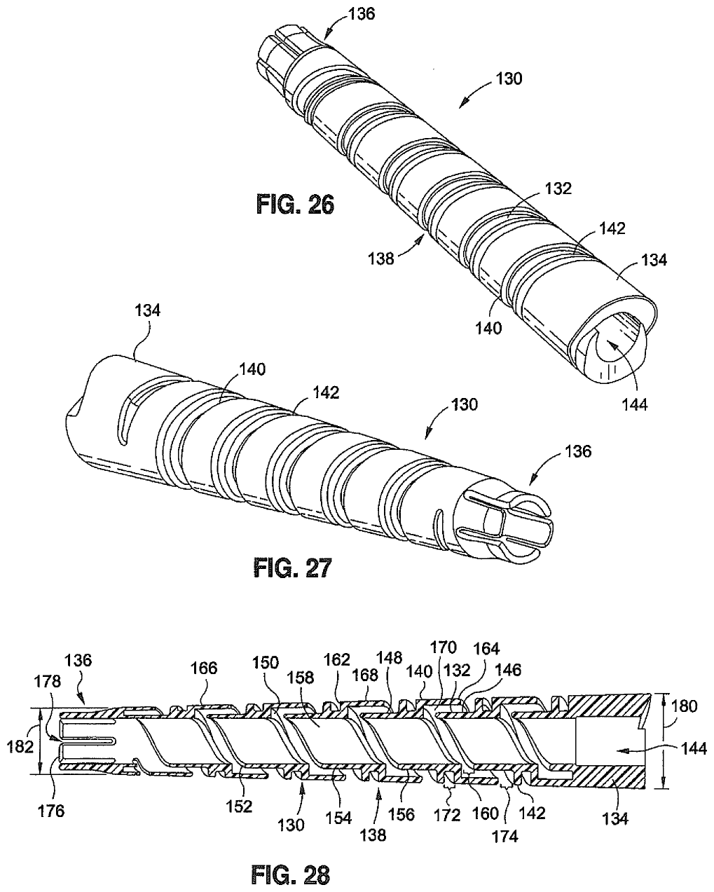

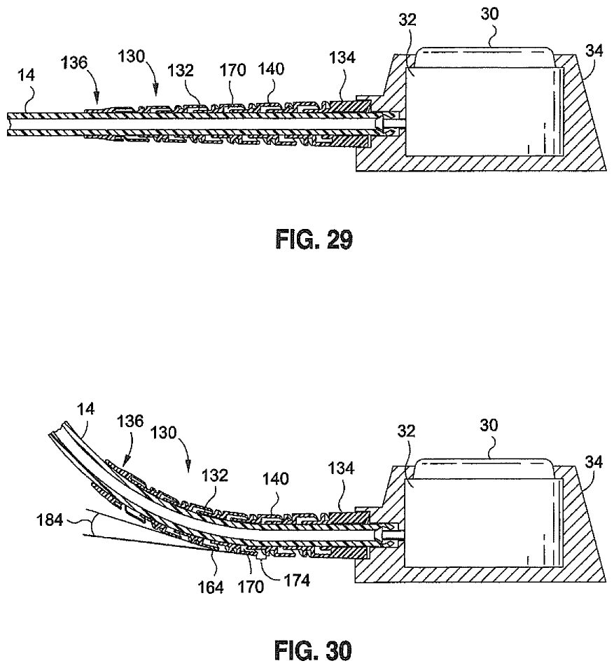

FIG. 26 illustrates a front perspective view of an embodiment of a shielding device 130 including a substantially spiral-shaped body portion 132 configured to wrap around the tube 14 (for example the tube 14 shown in FIG. 29). The shielding device 130 comprises a tapered cross-section, swept along a helix, to create a spiral geometry that is flexible, yet has no exposed gaps that allow a needle to penetrate the tube 14. The shielding device 130 has a connecting end 134 and a compliant end 136 and a middle portion 138 comprising the portion of the shielding device 130 that lies between the two ends 134, 136. An extended portion 140 of the shielding device 130 extends from the body portion 132 and has a spiral shape, to form an overlapping section of the shielding device 130. A ridge 142 extends from the body portion 132 and has a spiral shape. The wraps of the body portion 132 form a cylindrical shaped channel 144 through the interior of the shielding device 130 for the tube 14 to pass through.

FIG. 27 illustrates a rear perspective view of the shielding device 130 shown in FIG. 26.

FIG. 28 illustrates a side cross-sectional view of the shielding device 130 shown in FIGS. 26 and 27. The body portion 132 is a spiral or helical shaped structure that is configured to wrap multiple times around the tube 14 (for example the tube 14 shown in FIG. 29). The wraps are configured to be positioned adjacent to each other in succession along the length of the tube 14. Each wrap, for example a first wrap 146 and a second wrap 148, comprises a complete loop of the body portion 132 around the tube 14.

The body portion 132 has a leading edge 150 and a trailing edge 152 that are configured to spiral continuously around the tube 14. A flattened band-like middle section 154 of the body portion 132 connects the leading edge 150 to the trailing edge 152. The middle section 154 has a flattened outer surface 156. An inner surface 158 of the middle section 154 has a flattened shape, and is configured to face towards the tube 14.

The leading edge 150 and trailing edge 152 of adjacent wraps of the body portion 132 are separated by a gap 160. The gap 160 between adjacent wraps has a spiral shape due to the spiral configuration of the body portion 132. The gap 160 may have a length between approximately 0.020 to 0.050 inches, although this length may be varied as desired.

An extended portion 140 extends from the body portion 132 to cover the gap 160 positioned between adjacent wraps of the body portion 132. The extended portion 140 is structured to extend outward from the leading edge 150 of the body portion and extend over at least a portion of the gap 160, or entirely over the gap as shown in FIG. 28. The extended portion 140 may be structured to extend partially over the part of the body portion 132 forming an adjacent wrap. For example, FIG. 28 illustrates the extended portion 140 of the second wrap 148 extending over a part of the body portion 132 of the first wrap 146.

A trailing edge 162 of the extended portion 140 couples to the leading edge 150 of the body portion 132. The extended portion 140 is thus integrally connected with the body portion 132, as it is made from a unitary piece of material. A leading edge 164 of the extended portion 140 connects to the trailing edge 162 by a middle portion 166 of the extended portion 140. The middle portion 166 has a flattened outer surface 168. The middle portion 166 forms a spiral shaped hollow cavity 170 above the body portion 132. The cavity 170 is formed by an offset between the connection of the trailing edge 162 of the extended portion 140 and the leading edge 150 of the body portion 132. The gap 160 and a portion of the body portion 132 of an adjacent wrap are positioned within the cavity 170.

The spiral shaped ridge 142 comprises a continuous protrusion, or flange, extending from the body portion 132, and being positioned between the two edges 162, 164 of the extended portion 140. For example, FIG. 28 illustrates the ridge 142 of the first wrap 146 extending between the trailing edge 162 of the extended portion 140 of the first wrap 146 and the leading edge 164 of the extended portion 140 of the second wrap 148. The ridge 142 is separated from the trailing edge 162 of the extended portion 140 by a gap 172, and is separated from the leading edge 164 of the extended portion by a gap 174. The ridge 142 serves to strengthen and stiffen the local body portion 132 to which it is fixed.

The connecting end 134 of the shielding device 130 comprises a substantially solid piece of material, in which the extended portion 140 and the body portion 132 are integrated, and the extended portion 140 does not form a cavity 170. The connecting end 134 may be configured in any manner to allow the shielding device 130 to be firmly connected to the access port housing 34 (for example, the access port housing 34 shown in FIG. 29). For example, the connecting end 134 may be keyed to snap fit into the housing 34 (shown in FIG. 29).

The compliant end 136 of the shielding device 130 includes a fluted design having plurality of slats 176 separated by a plurality of slits 178. The slits 178 allows the slats 176 to flex, which provides a degree of flexible compliance for the shielding device 130, to prevent a tube 14 (for example, the tube 14 shown in FIG. 29) extending from the shielding device 130 from kinking as it exits the shielding device 130. The slits 178 and slats 176 enhance the strain relief properties of the shielding device 130.

The connecting end 134 preferably has a diameter 180 being larger than a diameter 182 of the compliant end 136. The middle portion 138 has a substantially tapered profile that smoothly transitions the diameter of the shielding device 130 from end 134 to end 136. In one embodiment, the diameter 180 of the connecting end 134 may be approximately 0.375 inches, and the diameter 182 of the compliant end 136 may be approximately 0.2 inches. The length of the shielding device 130 may be approximately 1.5 to 2 inches. The sizes of the diameters 180, 182, and length of the shielding device 130 may be varied as desired.

The diameter of the shielding device 130 preferably varies linearly from end 134 to end 136, as shown in FIG. 28. The height of the ridge 142 also decreases from end 134 to end 136. In addition, the thickness of the body portion 132 and extended portion 140 may decrease along successive wraps of the body portion 132 from the connecting end 134 to the compliant end 136. The body portion 132 may form as many wraps as desired around the tube 14, to accommodate various desired lengths of the shielding device 130. The shielding device 130 shown in FIG. 28 shows an exemplary number of five wraps of the body portion 132.

FIG. 29 illustrates a cross-section view of the shielding device 130 positioned along the length of the tube 14. The shielding device 130 entirely encircles the tube 14, to protect the tube 14 from errant needle sticks. The connecting end 134 is coupled to the access port housing 34 such that no portion of the tube 14 is exposed between the shielding device 130 and the housing 34. The connecting end 134 may be snapped or clipped to the housing 34. In one embodiment, the shielding device 130 is fixed to the tube 14 directly with an adhesive, for example silicone glue. In one embodiment, the shielding device 130 is overmolded on to the tube 14, or snapped on to the tube 14. In one embodiment, the shielding device 130 may be fixed to a flexible sleeve (not shown) that is slid over the tube 14 and either attached directly to the tube 14 or directly to the housing 34. In one embodiment, the shielding device 130 may be adhered directly to the housing 34, or overmolded to the housing 34. In one embodiment, the shielding device 130 is overmolded to the housing 34 and the tube 14.

The body portion 132 and extended portion 140 of the shielding device are both preferably made of a puncture resistant, yet flexible material. Such materials may include plastics, or polymers, including polysulfone. Any portion of the body portion 132 and/or extended portion 140 may be made of the same puncture resistant material. In addition, a portion of the shielding device 130 may not be made of puncture resistant material, if the shielding device 130 is still capable of providing equivalent shielding performance. Similar to the shielding device 18 discussed in relation to FIG. 7, the puncture resistant material may be capable of entirely blocking movement of an incoming syringe needle, or may merely resist penetration by a needle 36. For example, the puncture resistant material may deform when contacted by a syringe needle, but the energy required to pass through the shielding device 130 and contact the tube 14 may be great. The physician will notice the increased resistance and realize the needle 36 is not contacting the septum 30.

The shielding device 130 operates to protect the tube 14 from errant needle sticks by completely enclosing or covering the tube 14 from incident needles. The portion of tube exposed between adjacent wraps of the body portion 132 is covered by the extended portion 140. The cavity 170 and gap 174 between the leading edge 164 of the extended portion 140 of one wrap, and the ridge 142 of an adjacent wrap (referenced and discussed in relation to FIG. 28) are sized such that a standard sized syringe needle can not pass through the cavity 170, and potentially contact any exposed tube 14.

A benefit of the spiral design of the shielding device 130 is that the device 130 has a unitary construction, which allows the shielding device 130 to be placed over the tube 14 as a single unit. The shielding device 130 could therefore be quickly and easily installed to protect the tube 14. In addition, the shielding device 130 is made flexible, to allow the shielding device 130 to flex during implantation, or after implantation, to enhance a degree of comfort for the patient, similar to the shielding device 18 discussed in relation to FIG. 10. The use of gaps between adjacent wraps of the body portion 132 enhances the flexibility of the shielding device 130, as the grinding action between adjacent wraps that would normally result from a wrapped or spiral design, is reduced. Further, the tapered nature of the shielding device 130 acts enhances the strain relief properties of the shielding device 130, and enhances comfort for the patient and ease of implantation into the patient's body.

FIG. 30 illustrates the flexible properties of the shielding device 130. Adjacent wraps of the body portion 132 may move, or rotate, with respect to each other, forming an angle 184. The angle 184 may reach a maximal value of between seventy and ninety degrees, although this value may be varied as desired. The gap 174 between the ridge 142 and the leading edge 164 of the extended portion 140 is sized to prevent a standard sized syringe needle from passing through the cavity 170. The ridge 142 may be placed or sized to further prevent a syringe from passing through the cavity 170. The tapered profile of the shielding device 130 enhances the flexibility of the shielding device 130, as the portion of the device positioned closer to the compliant end 136 may be capable of flexing more than the portion of the device 130 positioned near the connecting end 134.

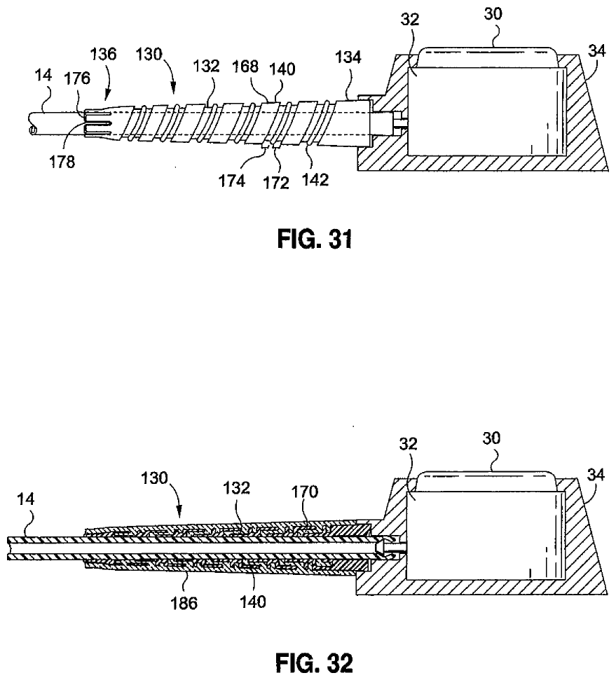

FIG. 31 illustrates a side view of the shielding device 130 in position along the tube 14. No portion of the tube 14 is exposed by the shielding device 130.

FIG. 32 illustrates an overmolding 186 that covers the shielding device 130. The overmolding 186 may be flexible in nature, and comprise an overmolding of an elastomeric material, such as silicone. The overmolding 186 fills in all gaps and cavities of the shielding device 130, forming shielding device 130 having no cavities, notwithstanding the cylindrical shaped channel 144 shown in FIG. 26. The overmolding 186 may enhance the biocompatibility of the device 130, by preventing local tissue from growing into the gaps 172, 174 (referenced in FIG. 31) of the shielding device 130. In addition, the elastomeric material may serve to enhance the overall comfort of the device for the patient. The shielding device 130 may be partially or fully over-molded with various materials, as desired. For example, a membrane of elastomeric material may be formed over the shielding device 130, configured similarly as the sheath 88 shown in FIG. 16.

The spiral shaped shielding device 130 is not limited to the embodiments shown in FIGS. 26-32, but may comprise any desired variation of shielding device 130 that wraps in a spiral and produces an equivalent result. For example, the dimensions of the spiral shaped shielding device 130 may be varied as desired. Various cross-section designs or configurations may be used, that are wrapped in a spiral, to produce varied flexibility and shielding device 130 performance characteristics. In addition, the construction of the compliant end 136 and connecting end 134 may be varied as desired. Further, the shape of the body portion 132, extended portion 140, and/or the cavity 170 formed by the extended portion 140 may appropriately be varied as desired. For example, the winding direction of the wraps may be reversed, or the leading edge 164 of the extended portion 140 may be configured to face the compliant end 136 of the shielding device 130.

In light of the shielding device 18, 130 embodiments disclosed above, the shielding device 18, 130 may be used in a gastric band system 10 that utilizes various components different from those discussed above. For example, a physician may insert the syringe needle to fill a pump reservoir, or maintain a fluid pressure in a mechanical pump system. In addition, a physician may insert a probe near the access port 16 to measure a local property of the gastric band system 10. The shielding device 18, 130 will still serve to protect the tube 14 from puncture in these systems that differ from the gastric band system 10 disclosed above.

Over-Molded Tubing Protector

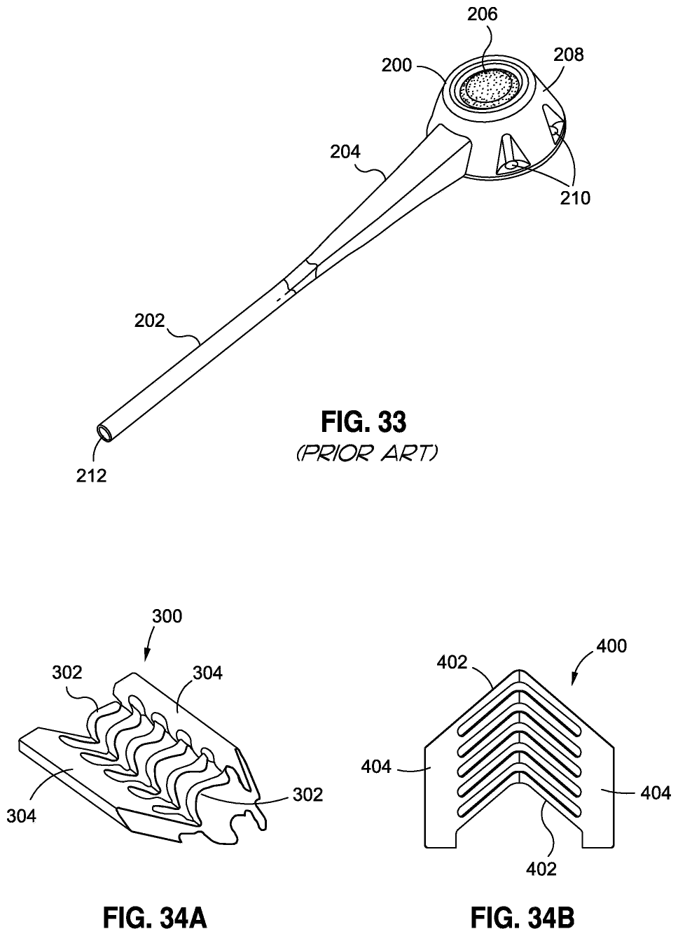

An adjustable (inflatable) gastric band can be effective to help an obese patient lose weight when the gastric band is properly tightened around the patient's esophageal-gastric junction (or around the top or cardia of the stomach) using fluid (saline) volume adjustments of the band. FIG. 33 shows a known (prior art) injection port or access port 200 that can be used to add or to remove fluid from the inflatable portion of a gastric band, thereby permitting a physician to adjust the volume of fluid for improved weight loss using a gastric band such as the LAP-BAND®. Injection port 200 typically is attached to a tubing or catheter 202 which fluidly connects injection port 200 to the gastric band (not shown). Tubing 202 includes a strain relief portion 204 adjacent injection port 200. Injection port 200 can have a needle penetrable septum 206 held in place by housing 208. Injection port 200 can be subdermally implanted (to the muscle or and/or fascia) and sutured in place by sutures looped through suture holes 210. Note that catheter 202 includes lumen or central channel 212, for fluid travel to and from the injection port.

As previously set forth an injection port, such as injection port 200, is accessed by a hypodermic needle inserted into eg septum 206 (see eg FIG. 1). The needle can miss septum 206 and instead puncture tubing 202 or strain relief 204, which can cause fluid leakage.

Two embodiments of a device to protect tubing 202 and/or strain relief 206 from needle puncture intended for septum 206 are shown in FIG. 34A as item 300 and in FIG. 34B as item 400, each for convenience called a tubing protector. Each of these tubing protectors can be made by an over molding process to thereby provide a single piece tubing protector. As indicated by FIG. 34 the tubing protector extends around three sides or around about 270 degrees of the circumference of the tubing or stain relief. The tubing protector does not extend around the full 360 degree circumference of the tube 202.

Tubing protector 300 comprises ribs 302 and tabs 304. Similarly, tubing protector 400 comprises ribs 402 and tabs 404. Tubing protector 300 comprises several (about 2 to about 15) tapered overlapping scale-like plastic shields (i.e. ribs 302) over-molded onto the silicone strain relief 204 to thereby protect the top of the tubing and strain relief from needle puncture. Ribs 302 are held together and properly spaced and oriented by the molding remnants (eg tabs 304) remaining from an over molding process.

Tubing protector 300 (and tubing protector 400) is made by an over molding process in which liquid silicone is placed onto and then molded around tubing 202 and/or stain relief 206. Tabs 304 are molding remnants that hold ribs 302 in position with respect to each other. Tabs 304 (molding remnants) ensure individual ribs 302 are spaced appropriately. After molding tabs 304 are broken off and discarded thus ribs 302 over-molded in place, thereby providing a tubing protector 300 securely attached onto strain relief 206. Tubing protector 300 can be made of silicone, such as a liquid silicone rubber (LSR, preferably a platinum cured LSR, more preferably MED-4850 available from Nusil Technology, LLC, Carpinteria, Calif. The over molding process is carried out at a pressure of between about 15 psi to 25 psi (preferably at about 20 psi), at a temperature of between about 120° C. to about 160° C., preferably about 140° C., for between about 8 minutes and about 12 minutes (preferably for about 10 minutes), resulting in the cured, silicone tubing protector 300 or in tubing protector 400.

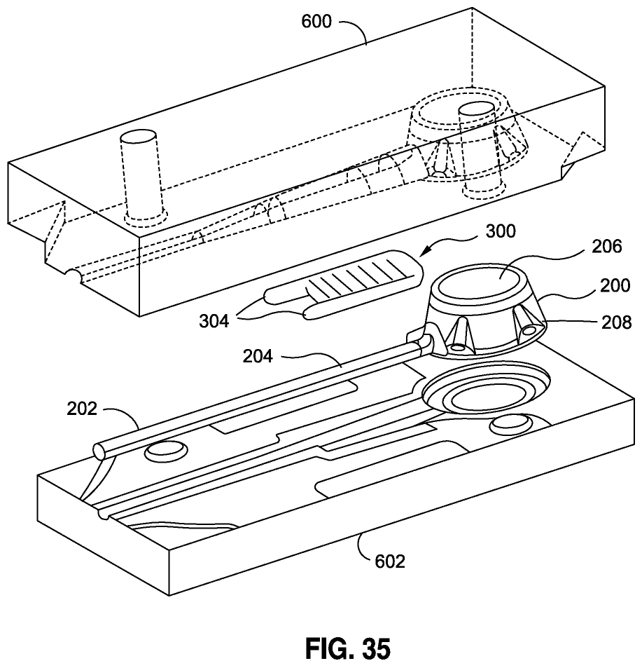

FIG. 35 shows is an elevation exploded view showing the result of the over molding process. Two metal (eg Aluminum or steel) molded halves 600 and 602 are prepared. Injection port 200 with tubing 202 and strain relief 206 is placed within and rests in its negative image in the two mold halves 600 and 602. Once in place, liquid LSR is added into the mold on top of strain relief 206, the two metal mold halves 600 and 602 are each pressed together by a hot press, and then the mold is allowed to cool before being opened. Tubing protectors 300 or 400 can also be made by a transfer mold process or by an injection molding process.

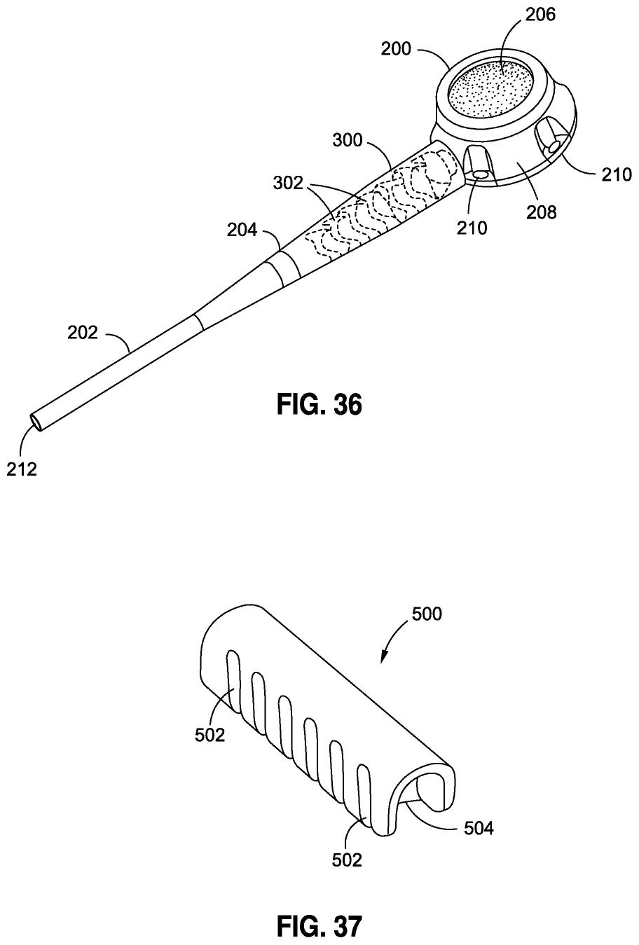

FIG. 36 is a top elevation view of showing tubing protector 300 (now removed from mold halves 600 and 602 and with tabs 304 removed) now securely in place over molded onto stain relief 206. As shown by FIG. 36 when tubing protector 300 is in place ribs 302 are inclined at an acute angle to the longitudinal axis of tubing 202 and strain relief 206, thereby permitting ribs 302 to overlap and thereby shield strain relief 206 from needle puncture.

FIG. 37 is also a top or front elevation view showing another embodiment of a tubing protector 500 which can be made by the over-molding process or by an encapsulation process. Tubing protector 500 has angular, U-shaped cuts or channels 502 which reduce use of LSR and can make it more flexible. Tubing 202 and strain relief 206 fit within lumen 504.

What is claimed is:

1. A shielding device for use to protect a tube from puncture, the tube extending from an access port of a gastric band system for the treatment of obesity, the shielding device comprising:
a plurality of arcuate or curved and overlapping parallel ribs made of a puncture resistant material, the ribs extending around substantially all the circumference of the tube adjacent the access port.

2. The shielding device of claim 1 wherein the ribs extend around about 270 degrees of the circumference of the tube.

3. The shielding device of claim 1, wherein the ribs are inclined at an acute angle to the longitudinal axis of the tube.

4. The shielding device of claim 1 wherein the shielding device is made by an over-molding process.

5. An over molding process for making a shielding device for use to protect a tube from puncture, the tube extending from an access port of a gastric band system for the treatment of obesity, the shielding device comprising a plurality of arcuate or curved and overlapping parallel ribs made of a puncture resistant material, the ribs extending around substantially all the circumference of the tube adjacent the access port, the over molding process comprising the following steps:
(a) adding liquid silicone to a two mold for the shielding device;
(b) applying a pressure of between about 15 psi to 25 psi to the mold;
(c) heating the liquid silicone to a temperature of between about 120 C to about 160 C, for between about 8 minutes and about 12 minutes;
(d) cooling the mold, and;
(e) removing the shielding device.
